SEQUENCE LISTING (1 sequence)

SEQ ID NO: 1 - protein, 8 aa, Artificial Sequence
  GS linker and hexahistidine tag for protein purification
GSHHHHHH

(19) United States
(12) Patent Application Publication
(10) Pub. No.: US 2007/0225344 A1
(43) Pub. Date: Sep. 27, 2007

(54) Sulfonamide derivatives to treat infection with hepatitis C virus

(76) Inventor: Ariamala Gopalsamy, Mahwah, NJ (US)

Correspondence Address: FITZPATRICK CELLA (WYETH), 30 ROCKEFELLER PLAZA, NEW YORK, NY, 10112-3800, US

(21) Appl. No.: 11/703,631

(22) Filed: Feb. 8, 2007

Related U.S. Application Data
(60) Provisional application No. 60/771,904, filed on Feb. 8, 2006.

(51) Int. Cl. A61K 31/426; A61K 31/41; A61K 31/4015; C07D 403/02; C07D 207/24

(52) U.S. Cl. 514/369; 514/381; 514/423; 514/424; 548/253; 548/530; 548/182; 548/542

(57) ABSTRACT

This invention is directed to compounds of formula (I): 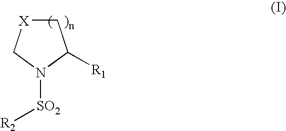
wherein R₁, R₂, X, and n are as defined herein, including all crystalline forms and pharmaceutically acceptable salts thereof, with the provisos that when X is CH₂, n is 1, and R₁ is —COOH, then R₂ cannot be 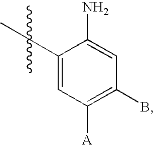
wherein A is CH₃—, CH₃CH₂— or a haloalkyl of 1 to 2 carbon atoms, and B is a halogen; and
when X is CH₂, n is 2, and R₁ is —COOH, then R₂ cannot be 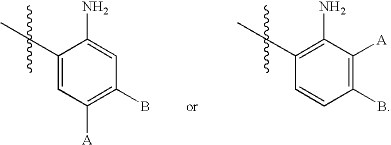 The invention is also directed to compositions containing compounds of the invention and methods of using the compounds to treat or prevent hepatitis C virus infections.

### CROSS REFERENCE TO RELATED APPLICATIONS

This application claims the benefit of U.S. Provisional Application No. 60/771,904, filed Feb. 8, 2006 the disclosure of which is incorporated by reference herein.

### FIELD OF THE INVENTION

A series of sulfonamide compounds are effective pharmaceuticals for the treatment of hepatitis C infection.

### BACKGROUND OF THE INVENTION

Hepatitis C is a common infection that can lead to chronic hepatitis, cirrhosis, liver failure, and hepatocellular carcinoma. Infection with the hepatitis C virus (HCV) leads to chronic hepatitis in at least 85% of cases, is the leading reason for liver transplantation, and is responsible for at least 10,000 deaths annually in the United States (Hepatology, 1997, 26 (Suppl. 1), 2S-10S).

The hepatitis C virus is a member of the Flaviviridae family, and the genome of HCV is a single-stranded linear RNA of positive sense (Hepatology, 1997, 26 (Suppl. 1), 11S-14S). HCV displays extensive genetic heterogeneity; at least 6 genotypes and more than 50 subtypes have been identified.

There is no effective vaccine to prevent HCV infection. The only therapy currently available is treatment with interferon-α (INF-α) or combination therapy of INF-α with the nucleoside analog ribavirin (Antiviral Chemistry and Chemotherapy, 1997, 8, 281-301). However, only about 40% of treated patients develop a sustained response, so there is a need for more effective anti-HCV therapeutic agents.

The HCV genome contains a number of non-structural proteins: NS2, NS3, NS4A, NS4B, NS5A, and NS5B (J. General Virology, 2000, 81, 1631-1648). NS5B is an RNA-dependent RNA polymerase that is essential for viral replication. Therefore, the inhibition of NS5B is a suitable target for the development of therapeutic agents.

U.S. Pat. No. 3,506,646 relates to compounds that are derivatives of 6-aminosulfonyl compounds, in particular 1,2,5-benzothiadiazepine 1,1-dioxides with fused heterocycles, the intermediates used to synthesize them, and their use as diuretic and hypotensive agents.

WO 98/08815 relates to substituted cyclic amine metalloprotease inhibitors.

Biorganic and medicinal chemistry, 1996, 837-850 describes 5H-pyrrolo[1,2-b][1,2,5]benzothiadiazepines (PBTDs) as a novel class of non-nucleoside reverse transcriptase inhibitors.

WO 03/043985 describes sulfonamides as peroxisome proliferator-activated receptor agonists.

WO 02/02554 describes sulfonyl-pyrrolidine derivatives useful for the treatment of neurological disorders.

### SUMMARY OF THE INVENTION

This invention relates to a series of sulfonamide derivatives, processes for their preparation, pharmaceutical compositions containing them, and their use in therapy. The compounds are believed to be useful in the treatment of hepatitis C by virtue of their ability to inhibit hepatitis C polymerase (NS5B).

This invention is directed to compounds of formula (I):

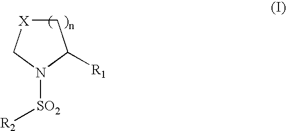

wherein:

R₁ is H, —COOH, —CO₂R₄, cyano, tetrazole, a straight chain alkyl of 1 to 6 carbon atoms optionally substituted with OH, amine or —COOH, an optionally substituted —C(O)-alkyl, or an optionally substituted —C(O)-aryl, wherein R₄ is an alkyl, aryl, cycloalkyl or heteroaryl, any of which may be optionally substituted;

R₂ is an aryl or a heteroaryl group optionally substituted with one to five substituents selected from the group consisting of halogen, —NO₂, —CN, —N₃, —CHO, —CF₃, —OCF₃, —R₃, —OR₃, S(O)ₘR₃, —NR₃R₃, NR₃S(O)ₘR₃, —NR₃C(O)R₃, —C(O)R₃, —C(O)OR₃, —C(O)NR₃R₃, —OC(O)R₃, —OC(O)OR₃, —OC(O)NR₃R₃, NR₃C(O)R₃, —NR₃C(O)OR₃, and —NR₃C(O)NR₃R₃, wherein m is 0, 1, or 2;

R₃ is H, an alkyl of 1-6 carbon atoms, a branched alkyl of 1-8 carbon atoms, a cycloalkyl of 3 to 6 carbon atoms, phenyl, a heteroaryl, an alkenyl of 2-6 carbon atoms, or an alkynyl of 2-6 carbon atoms;

X is CH₂, CHOR₃, or S; and

n is 1 or 2;

and all crystalline forms and pharmaceutically acceptable salts thereof, with the provisos that when X is CH₂, n is 1, and R₁ is —COOH, R₂ cannot be

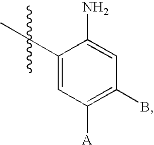

wherein:

A is CH₃—, CH₃CH₂— or a haloalkyl of 1 to 2 carbon atoms; and

B is a halogen; and

when X is CH₂, n is 2, and R₁ is —COOH, R₂ cannot be

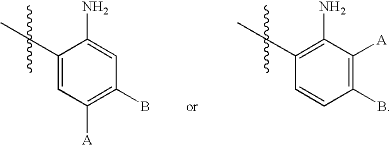

The present invention is also directed to compounds of formula (II):

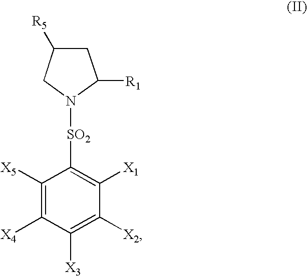

wherein:

R₁ is H, —COOH, —CN, tetrazole, —C(O)R₄, or a hydroxyalkyl of 1 to 4 carbon atoms, wherein R₄ is an alkyl of 1 to 4 carbon atoms or an optionally substituted phenyl;

R₅ is H, OH or —OCH₃; and

X₁-X₅ are independently H, a halogen, OH, NH₂, an alkyl of 1 to 4 carbon atoms, —NH—C(O)—R₃, wherein R₃ is an alkyl of 1 to 4 carbon atoms, an aryl, a cycloalkyl of 3 to 6 carbon atoms, or a heteroaryl;

and all crystalline forms or pharmaceutically acceptable salts thereof, with the provisos that when R₁ is —COOH, R₅ is H, X₃ is a halogen, and X₄ is —CH₃ or —CF₃, then X₁ cannot be NH₂; and

when R₁ is —COOH, R₅ is H, X₂ is —CH₃ or —CF₃, and X₃ is a halogen, then X₅ cannot be NH₂

Another aspect of the present invention are compounds of formula (III):

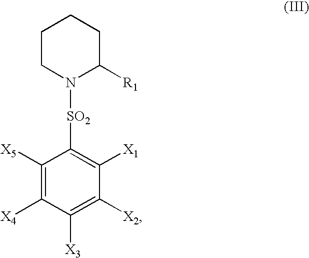

wherein:

R₁ is H, —COOH, —CN, tetrazole, —C(O)R₄, or a hydroxyalkyl of 1 to 4 carbon atoms, wherein R₄ is an alkyl of 1 to 4 carbon atoms or an optionally substituted phenyl; and

X₁-X₅ are independently H, a halogen, OH, NH₂, an alkyl of 1 to 4 carbon atoms, —NH—C(O)R₃, wherein R₃ is an alkyl of 1 to 4 carbon atoms, an aryl, a cycloalkyl of 3 to 6 carbon atoms or a heteroaryl;

and all crystalline forms and pharmaceutically acceptable salt thereof, with the provisos that when R₁ is —COOH, X₃ is a halogen, and X₄ is —CH₃ or —CH₂CH₃, then X₁ cannot be NH₂; and

when R₁ is —COOH, X₂ is —CH₃ or —CH₂CH₃, and X₃ is a halogen, then X₅ cannot be NH₂

The present invention is also directed to compounds of formula (IV):

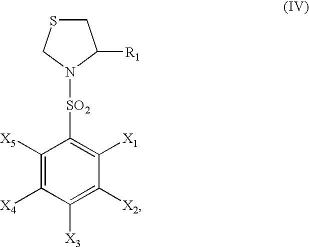

wherein:

R₁ is H, —COOH, —CN, tetrazole, —C(O)R₄, or a hydroxyalkyl of 1 to 4 carbon atoms, wherein R₄ is an alkyl of 1 to 4 carbon atoms or an optionally substituted phenyl; and

X₁ to X₅ are independently H, a halogen, OH, NH₂, an alkyl of 1 to 4 carbon atoms, —NH—C(O)R₃, wherein R₃ is an alkyl of 1 to 4 carbon atoms, an aryl, a cycloalkyl of 3 to 6 carbon atoms, or a heteroaryl;

and all crystalline forms and pharmaceutically acceptable salts thereof.

The present invention is also directed to pharmaceutical compositions comprising a compound of the present invention and a pharmaceutically acceptable carrier.

The present invention also includes methods of treating or preventing a hepatitis C infection in humans, comprising administering an effective amount of a compound of formula (Ia):

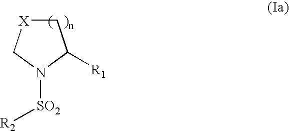

wherein:

R₁ is H, —COOH, —CO₂R₄, cyano, tetrazole, a straight chain alkyl of 1 to 6 carbon atoms optionally substituted with OH, amine, or —COOH, an optionally substituted —C(O)-alkyl, or an optionally substituted —(O)-aryl, wherein R₄ is an alkyl, aryl, cycloalkyl or a heteroaryl;

R₂ is an aryl or a heteroaryl group optionally substituted with one to five substituents selected from the group consisting of halogen, —NO₂, —CN, —N₃, —CHO, —CF₃, —OCF₃, —R₃, —OR₃, —S(O)ₘR₃, —NR₃R₃, —NR₃S(O)ₘR₃, —NR₃C(O)R₃, —C(O)R₃, —C(O)OR₃, —C(O)NR₃R₃, —OC(O)R₃, —OC(O)OR₃, —OC(O)NR₃R₃, NR₃C(O)R₃, —NR₃C(O)OR₃, and —NR₃C(O)NR₃R₃, wherein m is 0, 1, or 2;

R₃ is H, an alkyl of 1-6 carbon atoms, a branched alkyl of 1-8 carbon atoms, a cycloalkyl of 3 to 6 carbon atoms, phenyl, heteroaryl, an alkenyl of 2-6 carbon atoms, or an alkynyl of 2-6 carbon atoms;

X is CH₂, CHOR₃, or S; and

n is 1 or 2;

and all crystalline forms and pharmaceutically acceptable salts thereof.

### DETAILED DESCRIPTION OF THE INVENTION

For purposes of this invention the term “alkyl” includes straight chain moieties with a length of up to 12 carbon atoms, but preferably 1 to 6 carbon atoms, and more preferably 1 to 4 carbons. The term “alkyl” also includes branched moieties of 3 to 12 carbon atoms, but preferably 1 to 8 carbon atoms. The term “alkenyl” refers to a radical aliphatic hydrocarbon containing one double bond and includes both straight and branched alkenyl moieties of 2 to 7 carbon atoms. Such alkenyl moieties may exist in the E or Z configurations; the compounds of this invention include both configurations. The term “alkynyl” includes both straight chain and branched moieties containing 2 to 7 carbon atoms having at least one triple bond. The term “cycloalkyl” refers to alicyclic hydrocarbon groups having 3 to 12 carbon atoms and includes but is not limited to: cyclopropyl, cyclobutyl, cyclopentyl, cyclohexyl, cycloheptyl, norbornyl, or adamantyl. Preferably cycloalkyl groups are 3 to 6 carbon atoms.

For purposes of this invention the term “aryl” is defined as an aromatic hydrocarbon moiety having at least one aromatic ring, is mono-, bi- or tri-cyclic, and may be substituted or unsubstituted. An aryl group may be selected from but is not limited to: phenyl, α-naphthyl, β-naphthyl, biphenyl, anthryl, tetrahydronaphthyl, phenanthryl, fluorenyl, indanyl, biphenylenyl, acenaphthenyl, acenaphthylenyl, or phenanthrenyl. An aryl group may be optionally substituted with substituents selected from, but not limited to, the group consisting of alkyl, haloalkyl, acyl, alkoxycarbonyl, alkoxy, haloalkoxy, alkoxyalkyl, alkoxyalkoxy, cyano, halogen, hydroxy, nitro, trifluoromethyl, trifluoromethoxy, trifluoropropyl, amino, alkylamino, dialkylamino, dialkylaminoalkyl, hydroxyalkyl, alkoxyalkyl, alkylthio, mercapto, haloalkylthio, aryl, aryloxy, arylthio, heterocycloalkoxy, heterocycloalkylthio, —SO₃H, —SO₂NH₂, —O₂NHalkyl, —SO₂N(alkyl)₂, —CO₂H, CO₂NH₂, CO₂NHalkyl, and —CO₂N(alkyl)₂. Preferred substituents for aryl and heterocycloalkyl include: alkyl, halogen, amino, alkylamino, dialkylamino, trifluoromethyl, trifluoromethoxy, arylalkyl, and alkylaryl. Preferably an aryl group consists of 6 to 12 carbon atoms.

For purposes of this invention, the term “heteroaryl” is defined as: (1) an aromatic heterocyclic ring system (monocyclic or bicyclic) where the heteroaryl moieties are selected from furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline; and (2) a bicyclic aromatic heterocycle where a phenyl, pyridine, pyrimidine or pyridizine ring is: (a) fused to a 6-membered aromatic (unsaturated) heterocyclic ring having one nitrogen atom; (b) fused to a 5 or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Preferably a heteroaryl group consists of 2 to 9 carbon atoms.

For the purposes of this invention the term “hydroxyalkyl” is defined as an alkyl, as defined above, substituted with a hydroxyl group.

The compounds of this invention may contain an asymmetric carbon atom and one or more asymmetric centers, and may thus give rise to optical isomers and diastereomers. While shown without respect to stereochemistry in formulas (I), (II), (III), and (IV), the present invention includes all optical isomers and diastereomers, racemic and resolved, enantiomerically pure R and S stereoisomers, and other mixtures of the R and S stereoisomers and pharmaceutically acceptable salts thereof.

Pharmaceutically acceptable salts of the compounds of formulas (I), (II), (III), and (IV) with an acidic moiety can be formed from both organic and inorganic bases. For example, alkali metal salts such as sodium, lithium, and potassium, and N-tetraalkylammonium salts such as N-tetrabutylammonium salts. Similarly, when a compound of this invention contains a basic moiety, salts can be formed from organic and inorganic acids. For example, salts can be formed from acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids.

A preferred embodiment of the compounds of formula (I) is wherein X is CH₂ or S, especially where X is S and n is 1.

Another preferred embodiment of the compounds of formula (I) is wherein X is CH₂OR₃ and R₃ is H or CH₃, especially where n is 1.

Another preferred embodiment of the compounds of formula (I) is wherein R₁ is H, —COOH, —CN, tetrazole, —CH₂OH, —C(O)—CH₃ or —C(O)-phenyl, but even more preferred is where R₁ is —COOH.

Yet another preferred of the compounds of formula (I) is wherein R₂ is optionally substituted phenyl, especially where the phenyl ring is substituted by at least one substituent selected from OH, halogen, alkyl, amino, and —NR₃C(O)R₃

A preferred embodiment of the compounds of formula (II) is wherein R₅ is H, OH, or —OCH₃.

Another preferred embodiment of the compounds of formula (II) is wherein R₁ is —COOH.

Yet another preferred embodiment of the compounds of formula (II) is wherein at least one of X₁-X₅ is OH or —NH—C(O)—R₃.

A preferred embodiment of the compounds of formula (III) is wherein R₁ is —COOH.

Another preferred embodiment of the compounds of formula (III) is wherein X₁-X₅ are independently selected from H, NH₂, OH, halogen and alkyl, especially where at least one of X₁-X₅ is OH.

A preferred embodiment of the compounds of formula (IV) is wherein R₁ is —COOH or H.

Another preferred embodiment of the compounds of formula (IV) is wherein X₁-X₅ are independently selected from H, halogen, NH₂, alkyl and OH.

Preferred compounds of the present invention include:
2,4-dichloro-6-{[(2S)-2-(2H-tetrazol-5-yl)pyrrolidin-1-yl]sulfonyl}phenol;
(2S)-1-[(3,5-dichloro-2-hydroxyphenyl)sulfonyl]pyrrolidine-2-carbonitrile;
(4R)-4-hydroxy-1-[(3,4,5-trichloro-2-hydroxyphenyl)sulfonyl]-L-proline;
1-[(3,4,5-trichloro-2-hydroxyphenyl)sulfonyl]-L-proline;
1-[(3-bromo-5-chloro-2-hydroxyphenyl)sulfonyl]-L-proline;
(4R)-1-[(3,5-dibromo-2-hydroxyphenyl)sulfonyl]-4-hydroxy-L-proline;
(4R)-1-[(3-bromo-5-chloro-2-hydroxyphenyl)sulfonyl]-4-hydroxy-L-proline;
1-[(5-bromo-3-chloro-2-hydroxyphenyl)sulfonyl]-L-proline;
1-[(3,5-dibromo-2-hydroxyphenyl)sulfonyl]-L-proline;
(4R)-1-[(3,5-dichloro-2-hydroxyphenyl)sulfonyl]-4-hydroxy-L-proline;
1-[(3,5-dichloro-2-hydroxyphenyl)sulfonyl]-L-proline;
(4R)-1-[(3,5-dichloro-2-hydroxyphenyl)sulfonyl]-4-methoxy-L-proline;
1-[(3,4,5-trichloro-2-hydroxyphenyl)sulfonyl]-D-proline;
1-[(3-chloro-5-fluoro-2-hydroxyphenyl)sulfonyl]-L-proline;
(4S)-4-hydroxy-1-[(3,4,5-trichloro-2-hydroxyphenyl)sulfonyl]-D-proline;
1-{(2S)-1-[(3,5-dichloro-2-hydroxyphenyl)sulfonyl]pyrrolidin-2-yl}ethanone;
1-[(2-amino-5-chloro-4-methylphenyl)sulfonyl]-D-proline;
(4R)-1-[(3-chloro-5-fluoro-2-hydroxyphenyl)sulfonyl]-4-hydroxy-L-proline;
(4S)-1-[(2-amino-5-chloro-4-methylphenyl)sulfonyl]-4-hydroxy-D-proline;
1-[(2-amino-5-chloro-4-methylphenyl)sulfonyl]-L-proline; 2,4-dichloro-6-{[(2S)-2-(hydroxymethyl)pyrrolidin-1-yl]sulfonyl}phenol;
1-[(5-bromo-3-chloro-2-hydroxyphenyl)sulfonyl]-D-proline;
2,4-dichloro-6-(pyrrolidin-1-ylsulfonyl)phenol;
(4S)-1-[(2-amino-4-chloro-5-methylphenyl)sulfonyl]-4-hydroxy-D-proline;
(4R)-1-[(2-amino-5-chloro-4-methylphenyl)sulfonyl]-4-hydroxy-L-proline;
1-({5-chloro-4-methyl-2-[(2-thienylcarbonyl)amino]phenyl}sulfonyl)-L-proline; 1-[(3,5-dibromo-2-hydroxyphenyl)sulfonyl]-D-proline;
(4R)-1-[(2-amino-4-chloro-5-methylphenyl)sulfonyl]-4-hydroxy-L-proline;
1-{(2S)-1-[(3,5-dichloro-2-hydroxyphenyl)sulfonyl]pyrrolidin-2-yl}propan-1-one;
{(2S)-1-[(3,5-dichloro-2-hydroxyphenyl)sulfonyl]pyrrolidin-2-yl}(phenyl)methanone;
1-[(3,5-dichloro-2-hydroxyphenyl)sulfonyl]-D-proline;
1-[(2,4,6-trichlorophenyl)sulfonyl]-L-proline;
1-({5-chloro-2-[(cyclopropylcarbonyl)amino]-4-methylphenyl}sulfonyl)-L-proline;
1-{[2-(benzoylamino)-5-chloro-4-methylphenyl]sulfonyl}-L-proline;
1-[(3-chloro-4-methylphenyl)sulfonyl]-L-proline;
1-[(2,4,5-trichlorophenyl)sulfonyl]-L-proline;
(2S)-1-[(3,4,5-trichloro-2-hydroxyphenyl)sulfonyl]piperidine-2-carboxylic acid;
(2R)-1-[(3,4,5-trichloro-2-hydroxyphenyl)sulfonyl]piperidine-2-carboxylic acid;
(2S)-1-[(3-bromo-5-chloro-2-hydroxyphenyl)sulfonyl]piperidine-2-carboxylic acid;
(2R)-1-[(3-bromo-5-chloro-2-hydroxyphenyl)sulfonyl]piperidine-2-carboxylic acid;
(2S)-1-[(3-chloro-5-fluoro-2-hydroxyphenyl)sulfonyl]piperidine-2-carboxylic acid;
(2R)-1-[(3,5-dichloro-2-hydroxyphenyl)sulfonyl]piperidine-2-carboxylic acid;
(2S)-1-[(3,5-dichloro-2-hydroxyphenyl)sulfonyl]piperidine-2-carboxylic acid;
(2R)-1-[(2-amino-5-chloro-4-methylphenyl)sulfonyl]piperidine-2-carboxylic acid;
(4R)-3-[(3,4,5-trichloro-2-hydroxyphenyl)sulfonyl]-1,3-thiazolidine-4-carboxylic acid;
(4S)-3-[(3,4,5-trichloro-2-hydroxyphenyl)sulfonyl]-1,3-thiazolidine-4-carboxylic acid;
(4R)-3-[(3,5-dichloro-2-hydroxyphenyl)sulfonyl]-1,3-thiazolidine-4-carboxylic acid;
(4R)-3-[(3,5-dibromo-2-hydroxyphenyl)sulfonyl]-1,3-thiazolidine-4-carboxylic acid;
(4R)-3-[(3-chloro-5-fluoro-2-hydroxyphenyl)sulfonyl]-1,3-thiazolidine-4-carboxylic acid;
(4S)-3-[(3-bromo-5-chloro-2-hydroxyphenyl)sulfonyl]-1,3-thiazolidine-4-carboxylic acid;
(4S)-3-[(3,5-dichloro-2-hydroxyphenyl)sulfonyl]-1,3-thiazolidine-4-carboxylic acid;
2,4-dichloro-6-(1,3-thiazolidin-3-ylsulfonyl)phenol;
(4S)-3-[(2-amino-5-chloro-4-methylphenyl)sulfonyl]-1,3-thiazolidine-4-carboxylic acid;
(4R)-3-[(2-amino-4-chloro-5-methylphenyl)sulfonyl]-1,3-thiazolidine-4-carboxylic acid;
(4S)-3-[(2-amino-4-chloro-5-methylphenyl)sulfonyl]-1,3-thiazolidine-4-carboxylic acid;
(4R)-3-[(3-bromo-5-chloro-2-hydroxyphenyl)sulfonyl]-1,3-thiazolidine-4-carboxylic acid;
(4S)-3-[(3-chloro-5-fluoro-2-hydroxyphenyl)sulfonyl]-1,3-thiazolidine-4-carboxylic acid; and
(4R)-3-[(2-amino-5-chloro-4-methylphenyl)sulfonyl]-1,3-thiazolidine-4-carboxylic acid.

Compounds of the present invention inhibit the hepatitis C RNA-dependent RNA polymerase NS5B, and are therefore useful for the treatment of hepatitis C infection. The present invention accordingly provides a pharmaceutical composition that comprises a compound selected from formulas (I), (II), (III) and (IV) in combination or association with a pharmaceutically acceptable carrier. The compositions are preferably adapted for oral or subcutaneous administration. However, they may be adapted for other modes of administration. In order to obtain consistency of administration, it is preferred that a composition of the invention is in the form of a unit dose. Suitable unit dose forms include tablets, capsules and powders in sachets or vials. Such unit dose forms may contain from 0.1 to 100 mg of a compound of the invention, and preferably from 2 to 50 mg. Still further preferred unit dosage forms contain 5 to 25 mg of a compound of the present invention. The compounds of the present invention can be administered orally at a dose range of about 0.01 to 100 mg/kg, or preferably at a dose range of 0.1 to 10 mg/kg. Such compositions may be administered from 1 to 6 times a day, more usually from 1 to 4 times a day. The compositions of the invention may be formulated with conventional excipients, such as a filler, a disintegrating agent, a binder, a lubricant, a flavoring agent and the like.

The present invention further provides a compound of the invention for use as an active therapeutic substance. Compounds of formulas (Ia), (II), (III), and (IV) are of particular use for the treatment of infection with hepatitis C virus.

The present invention further provides a method of treating hepatitis C infection in humans, which comprises administering to the infected individual an effective amount of a compound of formulas (Ia), (II), (III), and (IV) or a pharmaceutical composition of the invention.

### General Synthetic Schemes for Preparation of Compounds of the Present Invention

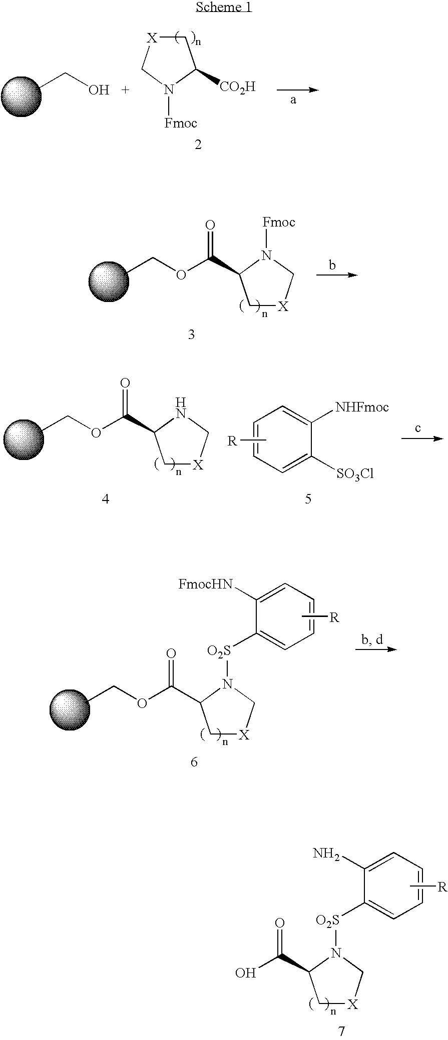 [0130] Reagents: (a) EDCI, HOBT, DIEA, DMF, RT, 6h; (b) 20% piperidine in DMF, RT, 20 min; (c) Pyridine, RT, ON; (d) 1:1 TFA-DCM, RT, 2h.

Scheme 1 shows how compounds of formula (I) can be prepared on a solid support using a resin, for example Wang resin. The Fmoc protected amino acid of interest 2 was attached to the resin using coupling agents, for example EDCI, HOBT in the presence of a base, and DIEA in a polar solvent. DMF may be used as the polar solvent, but one skilled in the art would be aware of other appropriate solvents. After washing the excess reagents and solvent, the amino acid attached to the resin was deprotected using base in DMF. Appropriate bases include alkylamine bases, for example piperidine, but skilled artisans would be aware of other possible bases to use. The free amino acid was reacted with the sulfonyl chloride of interest 5 in a solvent such as pyridine. Depending on the nature of the groups on the sulfonyl chloride, a deprotection step was employed before cleaving the product from the resin using trifluoroacetic acid and DCM. One skilled in the art would of be aware of the possible protecting groups that can be used to protect various functional groups from the acidic cleavage conditions.

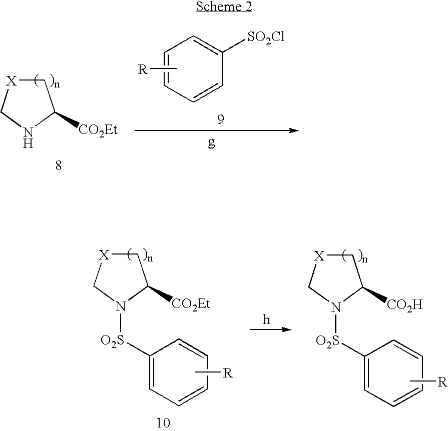

Reagents: (g) Pyridine, THF, RT, 8 h; (h) aq. NaOH, EtOH, RT, 8 h.

Alternately, the analogs can also be obtained by following Scheme 2. In this solution phase method, the amino acid ester of interest 8 was reacted with the sulfonyl chloride of choice 9 in pyridine, but other solvents may be used. The ester was hydrolyzed using a base like sodium hydroxide or lithium hydroxide to provide the required acid.

The reagents used in the preparation of the compounds of this invention can be either commercially obtained or can be prepared by standard procedures described in the literature. In accordance with this invention, the compounds described are produced by the reaction schemes shown above.

One skilled in the art would also understand how the methods of preparation detailed in Schemes 1 and 2 would also apply to compounds of formulas (II), (III) and (IV).

### Specific Synthesis of Compounds of the Present Invention

Examples 1, 2, 11 and 23 were synthesized in solution as shown in Scheme 2.

### EXAMPLE 1

### 2,4-dichloro-6-{[(2S)-2-(2H-tetrazol-5-yl)pyrrolidin-1-yl]sulfonyl}phenol

A mixture of (2S)-1-[(3,5-dichloro-2-hydroxyphenyl)sulfonyl]pyrrolidine-2-carbonitrile (37 mg, 0.12 mmol), sodium azide (24 mg, 0.36 mmol), and triethylamine hydrochloride (25 mg, 0.18 mmol) in 1.5 mL of DMF was stirred at 120° C. for 6 hours. The reaction was then cooled to room temperature, acidified by adding 2 mL of 1N HCl, and concentrated. 2,4-dichloro-6-{[(2S)-2-(2H-tetrazol-5-yl)pyrrolidin-1-yl]sulfonyl}phenol (31 mg, 71% yield) was obtained after reverse phase chromatography. ¹H NMR (DMSO-d6) δ 7.89 (d, J=3.5 Hz, 1H), 7.60 (d, J=3.5 Hz, 1H), 5.46 (dd, 1H), 3.56 (m, 1H), 3.33 (m, 1H), 2.30 (m, 1H), 1.96 (m, 3H). HRMS: calcd for C₁₁H₁₁Cl₂N₅O₃S, 364.00325; found (ESI-, [M−H]), 364.00349.

### EXAMPLE 2

### (2S)-1-[(3,5-dichloro-2-hydroxyphenyl)sulfonyl]pyrrolidine-2-carbonitrile

A mixture of (2S)-pyrrolidine-2-carbonitrile hydrochloride (34 mg, 0.2 mmol) and 3,5-dichloro-2-hydroxy-benzenesulfonyl chloride (57 mg, 0.22 mmol) in 2 mL of CH₂Cl₂/pyridine (1:1) was stirred at room temperature for 16 hours. Then the reaction mixture was concentrated and purified by reverse phase chromatography to give (2S)-1-(3,5-Dichloro-2-hydroxy-benzenesulfonyl)-pyrrolidine-2-carbonitrile (37 mg, 58% yield). ¹H NMR (DMSO-d6) δ 7.86 (d, J=2.8 Hz, 1H), 7.63 (d, J=2.8 Hz, 1H), 5.08 (br., 1H), 3.44(m, 1H), 3.18 (m, 1H), 2.54(m, 1H), 2.17 (m, 2H), 1.93 (m, 2H). HRMS: calcd for C₁₁H₁₀Cl₂N₂O₃S, 320.9862; found (ESI-, [M−H]), 320.98639.

Example 23 was prepared following the same procedure described for Example 2, except pyrrolidine was substituted for (2S)-pyrrolidine-2-carbonitrile hydrochloride.

### EXAMPLE 11

### 1-[(3,5-dichloro-2-hydroxyphenyl)sulfonyl]-L-proline

Step 1. To a solution of L-proline methyl ester hydrochloride (8.24 g; 50.1 mmol) in pyridine (100 mL) was added 3,5 dichloro-2-hydroxybenzene sulfonyl chloride (15 g; 57.3 mmol) at 0° C. The reaction mixture was warmed to room temperature and stirred for additional 4 h. The mixture was concentrated and purified by flash chromatography on silica gel (25% ethyl acetate in hexane) to yield 1-(3,5-Dichloro-2-hydroxy-benzenesulfonyl)-pyrrolidine-2-carboxylic acid methyl ester (8.6 g; 49%).

Step 2: The ester from step 1 was taken up in ethanol (175 mL) and 1 N sodium hydroxide was added and stirred overnight. The reaction mixture was then concentrated, diluted with water and extracted with ethyl acetate. The aqueous layer was acidified with 2N HCl to yield the desired compound as white solid (7.3 g; 89%). mp 107.4° C.; 1H NMR (CDCl3) δ 7.6 (d, 1H), 7.5 (d, 1H), 4.5 (dd, 1H), 3.4(m, 2H), 2.3 (m, 2H), 2.0 (m, 2H); MS (ESI) m/z 337.82; HRMS: calcd for C₁₁H₁₁Cl₂NO5S, 337.96622; found (ESI-, [M−H]), 337.96619.

Examples 3-10, 12-17, 18-19, 20-22, 24-58 were prepared in solid phase as described below for Example 20, using appropriately protected amino acids and sulfonyl chlorides, as shown in Scheme 1.

### EXAMPLE 20

### 1-[(2-amino-5-chloro-4-methylphenyl)sulfonyl]-L-proline

Step 1. 5-Chloro-2-(9H-fluoren-9-ylmethoxycarbonylamino)-4-methyl-benzenesulfonic acid. To a solution of 5-Chloro-2-amino-4-methyl-benzenesulfonic acid (3.3 g; 15.13 mmol) in water (40 mL) and sodium bicarbonate (2.8 g) was added a solution of 9-fluorenylmethoxycarbonyl chloride (4.3 g; 16.7 mmol) in dioxane (40 mL) at 0° C. The reaction mixture was stirred overnight and the volatiles were removed under reduced pressure. The aqueous layer was acidified with 2 N HCl, extracted with ethyl acetate, dried and concentrated. The residue was purified by flash column on silica gel (10% MeOH in EtOAc) to give a white solid (89%). ¹H NMR (DMSO-d6) δ 9.9 (s, 1H), 7.9 (d, J=8 Hz, 3H), 7.7 (d, J=8 Hz, 2H), 7.6 (s, 1H), 7.4 (m, 2H), 7.3 (m, 2H), 4.4 (d, J=7 Hz, 2H), 4.0 (t, J=7 Hz 1H), 2.3 (s, 3H).

Step 2. (4-Chloro-2-chlorosulfonyl-5-methyl-phenyl)-carbamic acid 9H-fluoren-9-ylmethyl ester. Sulfonic acid (3.0 g; 6.7 mmol) from step 1 was dissolved in 3 mL of DMF, and 2.5 mL of thionyl chloride was added dropwise at room temperature. The resulting solution was stirred at room temperature for an additional 4 hours and then quenched with ice and water. The white solid precipitate was filtered and dried, and used without further purification.

Step 3. Attachment of N-Fmoc-L-Proline to Wang Resin. Wang Resin (Ana Spec 100-200 mesh, 1% crosslinked; loading: 1.1 mmol/g; 5 g, 5.5 mmol) was swollen in anhydrous DMF (20 ml). A solution of N-Fmoc-L-Proline (7.4 g, 22 mmol), HOBT (3.37 g, 22 mmol), DMAP (268.8 mg, 2.2 mmol) and DIC (3.4 ml, 22 mmol) in anhydrous DMF (30 ml) was added to the resin. The mixture was shaken at room temperature on an orbital shaker overnight. The mixture was filtered and the resin washed with DMF (3×50 ml), MeOH (3×50 ml), CH₂Cl₂ (3×50 ml), and dried.

Step 4. Deprotection of Fmoc Group. The resin (5.5 mmol), prepared as described in step 1 above, was treated with a solution of 20% piperidine in DMF (2×50 ml, 10 min for the first time and 30 min for the second time) to remove the Fmoc protecting group from the resin. The mixture was filtered and the resin washed with DMF (3×50 ml), MeOH (3×50 ml), and CH₂Cl₂ (3×50 ml).

Step 5. Reaction with (4-Chloro-2-chlorosulfonyl-5-methyl-phenyl)-carbamic acid 9H-fluoren-9-ylmethyl ester. To the L-proline on Wang resin (5.5 mmol) was added a solution of (4-Chloro-2-chlorosulfonyl-5-methyl-phenyl)-carbamic acid 9H-fluoren-9-ylmethyl ester (5.1 g, 11 mmol) in 1:1 anhydrous CH₂Cl₂ and pyridine (50 ml). After shaking at room temperature overnight, the mixture was filtered, washed with MeOH (3×50 ml) and CH₂Cl₂ (5×50 ml).

Step 6. Deprotection of Fmoc group. The resin (5.5 mmol) obtained from step 5 was reacted again with a solution of 20% piperidine in DMF (2×50 ml, 10 min for the first time and 30 min for the second time). The mixture was filtered and the resin washed with DMF (3×50 ml), MeOH (3×50 ml), CH₂Cl₂ (3×50 ml), and dried.

Step 7. Cleavage from resin. The above resin was treated with 1:1 TFA:CH₂Cl₂ (50 ml) and was shaken at room temperature for 4 h. The mixture was filtered and the resin washed with CH₂Cl₂ (3×10 ml). The combined CH₂Cl₂ was concentrated and purified by HPLC. ¹H NMR (DMSO-d6) δ 7.4 (s, 1H), 6.8 (s, 1H), 6.3 (s, 2H), 4.3 (m, 1H), 3.2 (t, 2H), 2.2 (s, 3H), 2.1 (m, 1H), 1.9 (m, 1H), 1.65-1.8 (m, 2H). HRMS: calcd for C₁₂H₁₅ClN₂O₄S, 319.05139; found (ESI-FTMS, [M+H]), 319.05179.

### EXAMPLES

Examples of compounds of the present invention are listed in Table 1.

TABLE 1

| Example | |
|---|---|
| No. | Compound Name |
| 1 | 2,4-dichloro-6-{[(2S)-2-(2H-tetrazol-5-yl)pyrrolidin- |
| | 1-yl]sulfonyl}phenol |
| 2 | (2S)-1-[(3,5-dichloro-2-hydroxyphenyl)sulfonyl]pyrrolidine- |
| | 2-carbonitrile |
| 3 | (4R)-4-hydroxy-1-[(3,4,5-trichloro-2- |
| | hydroxyphenyl)sulfonyl]-L-proline |
| 4 | 1-[(3,4,5-trichloro-2-hydroxyphenyl)sulfonyl]-L-proline |
| 5 | 1-[(3-bromo-5-chloro-2-hydroxyphenyl)sulfonyl]-L-proline |
| 6 | (4R)-1-[(3,5-dibromo-2-hydroxyphenyl)sulfonyl]-4-hydroxy- |
| | L-proline |
| 7 | (4R)-1-[(3-bromo-5-chloro-2-hydroxyphenyl)sulfonyl]- |
| | 4-hydroxy-L-proline |
| 8 | 1-[(5-bromo-3-chloro-2-hydroxyphenyl)sulfonyl]-L-proline |
| 9 | 1-[(3,5-dibromo-2-hydroxyphenyl)sulfonyl]-L-proline |
| 10 | (4R)-1-[(3,5-dichloro-2-hydroxyphenyl)sulfonyl]-4-hydroxy- |
| | L-proline |
| 11 | 1-[(3,5-dichloro-2-hydroxyphenyl)sulfonyl]-L-proline |
| 12 | (4R)-1-[(3,5-dichloro-2-hydroxyphenyl)sulfonyl]-4-methoxy- |
| | L-proline |
| 13 | 1-[(3,4,5-trichloro-2-hydroxyphenyl)sulfonyl]-D-proline |
| 14 | 1-[(3-chloro-5-fluoro-2-hydroxyphenyl)sulfonyl]-L-proline |
| 15 | (4S)-4-hydroxy-1-[(3,4,5-trichloro-2- |
| | hydroxyphenyl)sulfonyl]-D-proline |
| 16 | 1-{(2S)-1-[(3,5-dichloro-2- |
| | hydroxyphenyl)sulfonyl]pyrrolidin-2-yl}ethanone |
| 17 | 1-[(2-amino-5-chloro-4-methylphenyl)sulfonyl]-D-proline |
| 18 | (4R)-1-[(3-chloro-5-fluoro-2-hydroxyphenyl)sulfonyl]-4- |
| | hydroxy-L-proline |
| 19 | (4S)-1-[(2-amino-5-chloro-4-methylphenyl)sulfonyl]-4- |
| | hydroxy-D-proline |
| 20 | 1-[(2-amino-5-chloro-4-methylphenyl)sulfonyl]-L-proline |
| 21 | 2,4-dichloro-6-{[(2S)-2-(hydroxymethyl)pyrrolidin-1- |
| | yl]sulfonyl}phenol |
| 22 | 1-[(5-bromo-3-chloro-2-hydroxyphenyl)sulfonyl]-D-proline |
| 23 | 2,4-dichloro-6-(pyrrolidin-1-ylsulfonyl)phenol |
| 24 | (4S)-1-[(2-amino-4-chloro-5-methylphenyl)sulfonyl]-4- |
| | hydroxy-D-proline |
| 25 | (4R)1-[(2-amino-5-chloro-4-methylphenyl)sulfonyl]-4- |
| | hydroxy-L-proline |
| 26 | 1-({5-chloro-4-methyl-2-[(2- |
| | thienylcarbonyl)amino]phenyl}sulfonyl)-L-proline |
| 27 | 1-[(3,5-dibromo-2-hydroxyphenyl)sulfonyl]-D-proline |
| 28 | (4R)-1-[(2-amino-4-chloro-5-methylphenyl)sulfonyl]-4- |
| | hydroxy-L-proline |
| 29 | 1-{(2S)-1-[(3,5-dichloro-2- |
| | hydroxyphenyl)sulfonyl]pyrrolidin-2-yl}propan-1-one |
| 30 | {(2S)-1-[(3,5-dichloro-2- |
| | hydroxyphenyl)sulfonyl]pyrrolidin-2-yl}(phenyl)methanone |
| 31 | 1-[(3,5-dichloro-2-hydroxyphenyl)sulfonyl]-D-proline |
| 32 | 1-[(2,4,6-trichlorophenyl)sulfonyl]-L-proline |
| 33 | 1-({5-chloro-2-[(cyclopropylcarbonyl)amino]-4- |
| | methylphenyl}sulfonyl)-L-proline |
| 34 | 1-{[2-(benzoylamino)-5-chloro-4- |
| | methylphenyl]sulfonyl}-L-proline |
| 35 | 1-[(3-chloro-4-methylphenyl)sulfonyl]-L-proline |
| 36 | 1-[(2,4,5-trichlorophenyl)sulfonyl]-L-proline |
| 37 | (2S)-1-[(3,4,5-trichloro-2- |
| | hydroxyphenyl)sulfonyl]piperidine-2-carboxylic acid |
| 38 | (2R)-1-[(3,4,5-trichloro-2- |
| | hydroxyphenyl)sulfonyl]piperidine-2-carboxylic acid |
| 39 | (2S)-1-[(3-bromo-5-chloro-2- |
| | hydroxyphenyl)sulfonyl]piperidine-2-carboxylic acid |
| 40 | (2R)-1-[(3-bromo-5-chloro-2- |
| | hydroxyphenyl)sulfonyl]piperidine-2-carboxylic acid |
| 41 | (2S)-1-[(3-chloro-5-fluoro-2- |
| | hydroxyphenyl)sulfonyl]piperidine-2-carboxylic acid |
| 42 | (2R)-1-[(3,5-dichloro-2- |
| | hydroxyphenyl)sulfonyl]piperidine-2-carboxylic acid |
| 43 | (2S)-1-[(3,5-dichloro-2- |
| | hydroxyphenyl)sulfonyl]piperidine-2-carboxylic acid |
| 44 | (2R)-1-[(2-amino-5-chloro-4- |
| | methylphenyl)sulfonyl]piperidine-2-carboxylic acid |
| 45 | (4R)-3-[(3,4,5-trichloro-2-hydroxyphenyl)sulfonyl]-1,3- |
| | thiazolidine-4-carboxylic acid |
| 46 | (4S)-3-[(3,4,5-trichloro-2-hydroxyphenyl)sulfonyl]-1,3- |
| | thiazolidine-4-carboxylic acid |
| 47 | (4R)-3-[(3,5-dichloro-2-hydroxyphenyl)sulfonyl]-1,3- |
| | thiazolidine-4-carboxylic acid |
| 48 | (4R)-3-[(3,5-dibromo-2-hydroxyphenyl)sulfonyl]-1,3- |
| | thiazolidine-4-carboxylic acid |
| 49 | (4R)-3-[(3-chloro-5-fluoro-2-hydroxyphenyl)sulfonyl]-1,3- |
| | thiazolidine-4-carboxylic acid |
| 50 | (4S)-3-[(3-bromo-5-chloro-2-hydroxyphenyl)sulfonyl]-1,3- |
| | thiazolidine-4-carboxylic acid |
| 51 | (4S)-3-[(3,5-dichloro-2-hydroxyphenyl)sulfonyl]-1,3- |
| | thiazolidine-4-carboxylic acid |
| 52 | 2,4-dichloro-6-(1,3-thiazolidin-3-ylsulfonyl)phenol |
| 53 | (4S)-3-[(2-amino-5-chloro-4-methylphenyl)sulfonyl]-1,3- |
| | thiazolidine-4-carboxylic acid |
| 54 | (4R)-3-[(2-amino-4-chloro-5-methylphenyl)sulfonyl]-1,3- |
| | thiazolidine-4-carboxylic acid |
| 55 | (4S)-3-[(2-amino-4-chloro-5-methylphenyl)sulfonyl]-1,3- |
| | thiazolidine-4-carboxylic acid |
| 56 | (4R)-3-[(3-bromo-5-chloro-2-hydroxyphenyl)sulfonyl]-1,3- |
| | thiazolidine-4-carboxylic acid |
| 57 | (4S)-3-[(3-chloro-5-fluoro-2-hydroxyphenyl)sulfonyl]-1,3- |
| | thiazolidine-4-carboxylic acid |
| 58 | (4R)-3-[(2-amino-5-chloro-4-methylphenyl)sulfonyl]-1,3- |
| | thiazolidine-4-carboxylic acid |

### Brief Description of Biological Test Procedure(s) and Text Summary of Results

The ability of the compounds of the present invention to inhibit hepatitis C polymerase was established by the following experimental procedure:

NS5B from the BK strain (1b subtype) is expressed in E. coli as a protein in which the 21 C-terminal amino acids are replaced with a short linker and a hexahistidine tag (GSHHHHHH). The purified protein is mixed with radioactive nucleotides and allowed to replicate a heteropolymeric RNA substrate, primed by an endogenous short hairpin, resulting in an approximately 760 nt product. The radioactive product is captured on a filter and quantitated after removal of the unincorporated nucleotides.

Reagents:

10 mM UTP (Promega # p116B)

10 mM ATP (Promega # p113B)

10 mM CTP (Promega # p114B)

10 mM GTP (Promega # p115B)

BSA 10 mg/ml NEB (100× at 10 mg/ml) #007-BSA

RNaseIn (Promega #N251X) 40 U/μl

³³P-GTP (NEN-easytides NEG/606H 3000 Ci/mmol, 370 MBq/ml, 10 mCi/ml)

Falcon polypropylene 96-well plates (Becton Dickinson #351190)

Millipore Multiscreen 96-well filtration plate #MADE NOB 50

Optiphase Supermix (Wallac) formulated by Fisher

Millipore Multiscreen liner for use in MicroBeta 1450-106 cassette (Wallac)

PerkinElmer #1450-433

1 M HEPES, pH 7.3

Amersham Pharmacia Biotec (US16924-500 ml)

1 M MgCl₂ (SIGMA #M1028)

DTT (solid) (SIGMA #D9779)

RNAse-free water (GIBCO-BRL #10977-023)

Dimethyl sulfoxide (Aldrich #27685-5)

Basilen Blue (Sigma, B5520)

0.5M EDTA, pH 8 (GIBCO-BRL #15575-020)

Dibasic sodium phosphate 7-hydrate (Na₂HPO₄.7H₂O; Baker#3824-07)

Phosphoric acid (Baker, #0262.02)

Further Reagent Preparation:

0.5 M Na Phosphate buffer. Per liter, weigh 134 g. Na₂HPO₄.7H₂O; add water to 900 ml. Adjust pH to 7.0 with phosphoric acid. Top off with water to 1 L.

Dilute nucleotides 1:1000 to 10 μM (GTP and CTP) or 1:100 to 100 μM (ATP and UTP) into RNAse-free water.

Procedure:

(1) Compounds 10 μl at 10 μg/ml in 15% DMSO

When starting from 100 μg/ml compound stock in 1% DMSO:

Dispense 5 μl 30% DMSO per well

Dispense 5 μl compound (100 μg/ml) per well.

When starting from 50 μg/ml compound stock in 15% DMSO:

Add 10 μl compound per well.

(2) Enzyme Mix:

| Stock | Final Conc. (in 50 μl assay volume) | | | | | |
|---|---|---|---|---|---|---|
| reactions | Per 20 μl mix (1 reaction) | | | | Per 600 | |
| DEPC H₂0 | | | 17.06 | μl | 10236 | μl |
| 1 M HEPES, | 20 | mM | 0.5 | μl | 300 | μl |
| pH 7.5 | | | | | | |
| 1 M MgCl₂ | 5 | mM | 0.25 | μl | 150 | μl |
| 100 mM DTT | 1 | mM | 0.5 | μl | 300 | μl |
| 100 μM UTP | 0.5 | μM | 0.25 | μl | 150 | μl |
| 100 μM ATP | 1 | μM | 0.5 | μl | 300 | μl |
| 10 μM CTP | 0.08 | μM | 0.4 | μl | 240 | μl |
| 10 μM GTP | 0.025 | μM | 0.125 | μl | 75 | μl |
| BSA, 10 mg/ml | 0.05 | mg/ml | 0.25 | μl | 150 | μl |
| HCV RdRp | 24 | nM | 0.16 | μl | 96 | μl |
| NS5B d21BK | | | | | | |
| (500 ug/ml | | | | | | |
| or ˜7.5 uM) | | | | | | |
| | | | Total: 20 | μl | 12 | ml |

Add 20 μl enzyme mix into each well of the assay plate. Incubate compound and enzyme at room temperature for 15 minutes.

(3) Template Mix—Prepare Ahead.

Spin down a tube of RNA (5 μg/tube stored in 75% ethanol and 0.3 M sodium acetate) in a microcentrifuge for 20 min. at 4° C. One tube is enough for 1 to 1½ plates. Remove as much ethanol from the tube as possible by inverting the tube. Be gentle, pellet RNA may not adhere to the tube. Vacuum dry the RNA. Resuspend the RNA by adding 1 ml of DEPC water, close the cap of the tube tightly. To dissolve RNA, incubate RNA solution on ice for ˜60 min. and gently vortex. Spin briefly to ensure all RNA solution is down to the bottom of the tube before opening cap. Gently transfer RNA solution into a 5 ml or larger tube. Add another 3 ml of DEPC water (total 4 ml of volume).

Add the following volumes of reagents.

| | Final | | | | | |
|---|---|---|---|---|---|---|
| | concentration | | | | | |
| | Per 600 | | Per 20 | | | |
| Stock | reactions | | μl mix | | (1 reaction) | |
| RNAse-free water | | | 2.98 | μl | 1788 | μl |
| Hepes, 1M | 20 | mM | 0.5 | μl | 300 | μl |
| Rnase Inhibitor | 0.4 | U/μl | 0.5 | μl | 300 | μl |
| (40 (1 reaction)/μl) | | | | | | |
| ³³P-GTP 3000 Ci/mmol, | 0.025 | μM | 0.0125 | μl | 7.5 | μl |
| 10 μCi/μl (3.3 μM) | | | | | | |
| POF | 3 | nM | 16 | μl | 9600 | μl |

Add 20 μl template mix per reaction (i.e. 20 ng of POF per reaction or ˜3 nM)

(4) Incubate reaction at room temperature (22-25° C.) for 2 hours.

(5) Stop reaction by adding 50 μl of 170 mM EDTA.

Final concentration of EDTA is 85 mM.

(6) Prewet filters of Millipore Multiscreen filter plate by adding 200 μl of 0.5 M sodium phosphate buffer, pH 7.0 into each well. Let stand at room temperature for 2-3 min.

(7) Place the Multiscreen filter plate onto a Millipore Manifold and turn on vacuum to allow buffer to flow through. Turn off vacuum. Transfer 80 μl of the reaction product into each well of the filter plate. Let stand for 2-3 min. Turn on vacuum to filter reaction product.

(8) Turn off vacuum. Add 200 μl of 0.5 M sodium phosphate buffer, pH 7.0 into each well to wash filter. Turn on vacuum.

Repeat step (8) three more times.

(9) Remove polypropylene bottom. Spot dry filter at the bottom with paper towel. Air dry filter plate on a bench for 1 hr. Add 40 μl Super Mix scintillant. Seal top of the plate with a tape. Place plate into a Packard carrier or MicroBeta carrier.

(10) Count plate using a Packard Topcount or MicroBeta counter. Program 10 for ³³P in Top count or ³³P program in micro-beta.

Analysis of Results

Percent inhibition is calculated after background subtraction as a percent reduction of activity relative to the positive control (average value of the plate excluding the negative controls). For the primary screen hits were chosen as showing ≧75% inhibition.

Table 2 shows the in vitro inhibitory activity for the compounds of the present invention towards HCV polymerase.

TABLE 2

| | HCV Polymerase |
|---|---|
| Example No. | Median IC50 (μM) |
| 1 | 0.56 |
| 2 | 1.04 |
| 3 | 0.07 |
| 4 | 0.08 |
| 5 | 0.26 |
| 6 | 0.46 |
| 7 | 0.47 |
| 8 | 0.40 |
| 9 | 0.56 |
| 10 | 0.67 |
| 11 | 0.89 |
| 12 | 0.91 |
| 13 | 1.17 |
| 14 | 2.05 |
| 15 | 1.22 |
| 16 | 2.62 |
| 17 | 4.70 |
| 18 | 3.28 |
| 19 | 4.05 |
| 20 | 5.95 |
| 21 | 6.10 |
| 22 | 4.81 |
| 23 | 4.85 |
| 24 | 5.10 |
| 25 | 7.65 |
| 26 | 6.87 |
| 27 | 7.00 |
| 28 | 10.00 |
| 29 | 7.29 |
| 30 | 7.52 |
| 31 | 6.45 |
| 32 | 9.60 |
| 33 | 8.68 |
| 34 | 10.11 |
| 35 | 10.25 |
| 36 | 21.90 |
| 37 | 0.08 |
| 38 | 0.38 |
| 39 | 0.45 |
| 40 | 1.16 |
| 41 | 1.89 |
| 42 | 2.01 |
| 43 | 7.10 |
| 44 | 7.90 |
| 45 | 0.05 |
| 46 | 0.26 |
| 47 | 0.24 |
| 48 | 0.46 |
| 49 | 2.13 |
| 50 | 0.87 |
| 51 | 1.71 |
| 52 | 2.35 |
| 53 | 3.05 |
| 54 | 4.75 |
| 55 | 7.50 |
| 56 | 11.20 |
| 57 | 14.40 |
| 58 | 1.98 |

What is claimed is:

1. A compound of formula (I): 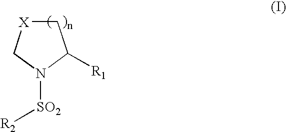
wherein:
R₁ is H, —COOH, —CO₂R₄, cyano, tetrazole, a straight chain alkyl of 1 to 6 carbon atoms optionally substituted with OH, amine or —COOH, an optionally substituted —C(O)-alkyl, or an optionally substituted —C(O)-aryl, wherein R₄ is an alkyl, aryl, cycloalkyl or heteroaryl, any of which may be optionally substituted;
R₂ is an aryl or a heteroaryl group optionally substituted with one to five substituents selected from the group consisting of halogen, —NO₂, —CN, —N₃, —CHO, —CF₃, —OCF₃, —R₃, —OR₃, —S(O)ₘR₃, —NR₃R₃, —NR₃S(O)ₘR₃, —NR₃C(O)R₃, —C(O)R₃, —C(O)OR₃, —C(O)NR₃R₃, —OC(O)R₃, —OC(O)OR₃, —OC(O)NR₃R₃, NR₃C(O)R₃, —NR₃C(O)OR₃, and —NR₃C(O)NR₃R₃, wherein m is 0, 1, or 2;
R₃ is H, an alkyl of 1-6 carbon atoms, a branched alkyl of 1-8 carbon atoms, a cycloalkyl of 3 to 6 carbon atoms, phenyl, a heteroaryl, an alkenyl of 2-6 carbon atoms, or an alkynyl of 2-6 carbon atoms;
X is CH₂, CHOR₃, or S; and
n is 1 or 2;
and all crystalline forms and pharmaceutically acceptable salts thereof, with the provisos that when X is CH₂, n is 1, and R₁ is —COOH, R₂ cannot be 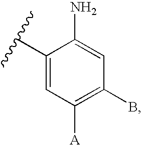
wherein:
A is CH₃—, CH₃CH₂— or a haloalkyl of 1 to 2 carbon atoms; and
B is a halogen; and
when X is CH₂, n is 2, and R₁ is —COOH, then R₂ cannot be

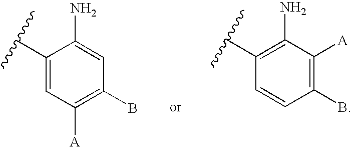

2. The compound of claim 1, wherein R₂ is an optionally substituted phenyl.

3. The compound of claim 1, wherein X is CH₂.

4. The compound of claim 1, wherein X is CH₂OR₃ and R₃ is H or CH₃.

5. The compound of claim 4, wherein n is 1.

6. The compound of claim 1, wherein X is S.

7. The compound of claim 6, wherein n is 1.

8. The compound of claim 1, wherein R₁ is H, —COOH, —CN, tetrazole, —CH₂OH, —C(O)—CH₃, or —C(O)-phenyl.

9. The compound of claim 8, wherein R₁ is —COOH.

10. The compound of claim 2, wherein the phenyl ring is substituted by at least one substituent selected from OH, halogen, alkyl, amino, and —NR₃C(O)R₃.

11. The compound of claim 3, wherein R₂ cannot be phenyl substituted with NH₂, an alkyl, and a halogen when R₁ is —COOH.

12. A compound of formula (II): 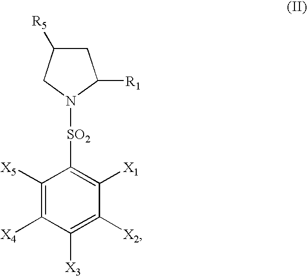
wherein:
R₁ is H, —COOH, —CN, tetrazole, —C(O)R₄, or a hydroxyalkyl of 1 to 4 carbon atoms, wherein R₄ is an alkyl of 1 to 4 carbon atoms or an optionally substituted phenyl;
R₅ is H, OH or —OCH₃; and
X₁-X₅ are independently H, a halogen, OH, NH₂, an alkyl of 1 to 4 carbon atoms, —NH—C(O)—R₃, wherein R₃ is an alkyl of 1 to 4 carbon atoms, an aryl, a cycloalkyl of 3 to 6 carbon atoms, or a heteroaryl;
and all crystalline forms or pharmaceutically acceptable salts thereof, with the provisos that when R₁ is —COOH, R₅ is H, X₃ is a halogen, and X₄ is —CH₃ or —CF₃, then X₁ cannot be NH₂, and
when R₁ is —COOH, R₅ is H, X₂ is —CH₃ or —CF₃, and X₃ is a halogen, then X₅ cannot be NH₂.

13. The compound of claim 12, wherein R₅ is H.

14. The compound of claim 12, wherein R₅ is OH.

15. The compound of claim 12, wherein R₅ is —OCH₃.

16. The compound of claim 12, wherein R₁ is —COOH.

17. The compound of claim 12, wherein at least one of X₁-X₅ is OH.

18. The compound of claim 12, wherein at least one of X₁-X₅ is —NH—C(O)—R₃.

19. A compound of formula (III): 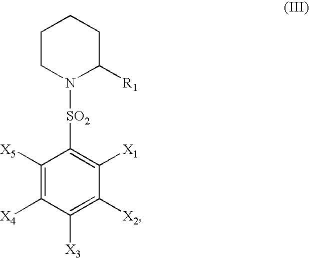
wherein:
R₁ is H, —COOH, —CN, tetrazole, —C(O)R₄, or a hydroxyalkyl of 1 to 4 carbon atoms, wherein R₄ is an alkyl of 1 to 4 carbon atoms or an optionally substituted phenyl; and
X₁-X₅ are independently H, a halogen, OH, NH₂, an alkyl of 1 to 4 carbon atoms, —NH—C(O)R₃, wherein R₃ is an alkyl of 1 to 4 carbon atoms, an aryl, a cycloalkyl of 3 to 6 carbon atoms or a heteroaryl;
and all crystalline forms and pharmaceutically acceptable salt thereof, with the provisos that when R₁ is —COOH, X₃ is a halogen, and X₄ is —CH₃ or —CH₂CH₃, then X₁ cannot be NH₂; and
when R₁ is —COOH, X₂ is —CH₃ or —CH₂CH₃, and X₃ is a halogen, then X₅ cannot be NH₂.

20. The compound of claim 19, wherein R₁ is —COOH.

21. The compound of claim 19, wherein X₁-X₅ are independently selected from H, NH₂, OH, halogen, and alkyl.

22. The compound of claim 21, wherein at least one of X₁-X₅ is OH.

23. A compound of formula (IV): 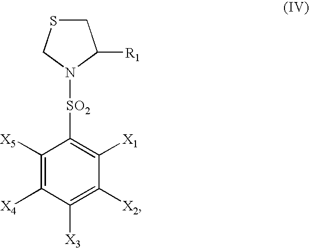
wherein:
R₁ is H, —COOH, —CN, tetrazole, —C(O)R₄, or a hydroxyalkyl of 1 to 4 carbon atoms, wherein R₄ is an alkyl of 1 to 4 carbon atoms or an optionally substituted phenyl; and
X₁ to X₅ are independently H, a halogen, OH, NH₂, an alkyl of 1 to 4 carbon atoms, —NH—C(O)R₃, wherein R₃ is an alkyl of 1 to 4 carbon atoms, an aryl, a cycloalkyl of 3 to 6 carbon atoms, or a heteroaryl;
and all crystalline forms and pharmaceutically acceptable salts thereof.

24. The compound of claim 23, wherein R₁ is —COOH or H.

25. The compound of claim 23, wherein X₁-X₅ are independently selected from H, halogen, NH₂, alkyl, and OH.

26. The compound of claim 1, wherein the compound is selected from:
2,4-dichloro-6-{[(2S)-2-(2H-tetrazol-5-yl)pyrrolidin-1-yl]sulfonyl}phenol;
(2S)-1-[(3,5-dichloro-2-hydroxyphenyl)sulfonyl]pyrrolidine-2-carbonitrile;
(4R)-4-hydroxy-1-[(3,4,5-trichloro-2-hydroxyphenyl)sulfonyl]-L-proline;
1-[(3,4,5-trichloro-2-hydroxyphenyl)sulfonyl]-L-proline;
1-[(3-bromo-5-chloro-2-hydroxyphenyl)sulfonyl]-L-proline;
(4R)-1-[(3,5-dibromo-2-hydroxyphenyl)sulfonyl]-4-hydroxy-L-proline;
(4R)-1-[(3-bromo-5-chloro-2-hydroxyphenyl)sulfonyl]-4-hydroxy-L-proline;
1-[(5-bromo-3-chloro-2-hydroxyphenyl)sulfonyl]-L-proline;
1-[(3,5-dibromo-2-hydroxyphenyl)sulfonyl]-L-proline;
(4R)-1-[(3,5-dichloro-2-hydroxyphenyl)sulfonyl]-4-hydroxy-L-proline;
1-[(3,5-dichloro-2-hydroxyphenyl)sulfonyl]-L-proline;
(4R)-1-[(3,5-dichloro-2-hydroxyphenyl)sulfonyl]-4-methoxy-L-proline;
1-[(3,4,5-trichloro-2-hydroxyphenyl)sulfonyl]-D-proline;
1-[(3-chloro-5-fluoro-2-hydroxyphenyl)sulfonyl]-L-proline;
(4S)-4-hydroxy-1-[(3,4,5-trichloro-2-hydroxyphenyl)sulfonyl]-D-proline;
1-{(2S)-1-[(3,5-dichloro-2-hydroxyphenyl)sulfonyl]pyrrolidin-2-yl}ethanone;
1-[(2-amino-5-chloro-4-methylphenyl)sulfonyl]-D-proline;
(4R)-1-[(3-chloro-5-fluoro-2-hydroxyphenyl)sulfonyl]-4-hydroxy-L-proline;
(4S)-1-[(2-amino-5-chloro-4-methylphenyl)sulfonyl]-4-hydroxy-D-proline;
1-[(2-amino-5-chloro-4-methylphenyl)sulfonyl]-L-proline;
2,4-dichloro-6-{[(2S)-2-(hydroxymethyl)pyrrolidin-1-yl]sulfonyl}phenol;
1-[(5-bromo-3-chloro-2-hydroxyphenyl)sulfonyl]-D-proline;
2,4-dichloro-6-(pyrrolidin-1-ylsulfonyl)phenol;
(4S)-1-[(2-amino-4-chloro-5-methylphenyl)sulfonyl]-4-hydroxy-D-proline;
(4R)-1-[(2-amino-5-chloro-4-methylphenyl)sulfonyl]-4-hydroxy-L-proline;
1-({5-chloro-4-methyl-2-[(2-thienylcarbonyl)amino]phenyl}sulfonyl)-L-proline;
1-[(3,5-dibromo-2-hydroxyphenyl)sulfonyl]-D-proline;
(4R)-1-[(2-amino-4-chloro-5-methylphenyl)sulfonyl]-4-hydroxy-L-proline;
1-{(2S)-1-[(3,5-dichloro-2-hydroxyphenyl)sulfonyl]pyrrolidin-2-yl}propan-1-one;
{(2S)-1-[(3,5-dichloro-2-hydroxyphenyl)sulfonyl]pyrrolidin-2-yl}(phenyl)methanone;
1-[(3,5-dichloro-2-hydroxyphenyl)sulfonyl]-D-proline;
1-[(2,4,6-trichlorophenyl)sulfonyl]-L-proline; 1-({5-chloro-2-[(cyclopropylcarbonyl)amino]-4-methylphenyl}sulfonyl)-L-proline;
1-{[2-(benzoylamino)-5-chloro-4-methylphenyl]sulfonyl}-L-proline;
1-[(3-chloro-4-methylphenyl)sulfonyl]-L-proline; 1-[(2,4,5-trichlorophenyl)sulfonyl]-L-proline;
(2S)-1-[(3,4,5-trichloro-2-hydroxyphenyl)sulfonyl]piperidine-2-carboxylic acid;
(2R)-1-[(3,4,5-trichloro-2-hydroxyphenyl)sulfonyl]piperidine-2-carboxylic acid;
(2S)-1-[(3-bromo-5-chloro-2-hydroxyphenyl)sulfonyl]piperidine-2-carboxylic acid;
(2R)-1-[(3-bromo-5-chloro-2-hydroxyphenyl)sulfonyl]piperidine-2-carboxylic acid;
(2S)-1-[(3-chloro-5-fluoro-2-hydroxyphenyl)sulfonyl]piperidine-2-carboxylic acid;
(2R)-1-[(3,5-dichloro-2-hydroxyphenyl)sulfonyl]piperidine-2-carboxylic acid;
(2S)-1-[(3,5-dichloro-2-hydroxyphenyl)sulfonyl]piperidine-2-carboxylic acid;
(2R)-1-[(2-amino-5-chloro-4-methylphenyl)sulfonyl]piperidine-2-carboxylic acid;
(4R)-3-[(3,4,5-trichloro-2-hydroxyphenyl)sulfonyl]-1,3-thiazolidine-4-carboxylic acid;
(4S)-3-[(3,4,5-trichloro-2-hydroxyphenyl)sulfonyl]-1,3-thiazolidine-4-carboxylic acid;
(4R)-3-[(3,5-dichloro-2-hydroxyphenyl)sulfonyl]-1,3-thiazolidine-4-carboxylic acid;
(4R)-3-[(3,5-dibromo-2-hydroxyphenyl)sulfonyl]-1,3-thiazolidine-4-carboxylic acid;
(4R)-3-[(3-chloro-5-fluoro-2-hydroxyphenyl)sulfonyl]-1,3-thiazolidine-4-carboxylic acid;
(4S)-3-[(3-bromo-5-chloro-2-hydroxyphenyl)sulfonyl]-1,3-thiazolidine-4-carboxylic acid;
(4S)-3-[(3,5-dichloro-2-hydroxyphenyl)sulfonyl]-1,3-thiazolidine-4-carboxylic acid;
2,4-dichloro-6-(1,3-thiazolidin-3-ylsulfonyl)phenol;
(4S)-3-[(2-amino-5-chloro-4-methylphenyl)sulfonyl]-1,3-thiazolidine-4-carboxylic acid;
(4R)-3-[(2-amino-4-chloro-5-methylphenyl)sulfonyl]-1,3-thiazolidine-4-carboxylic acid;
(4S)-3-[(2-amino-4-chloro-5-methylphenyl)sulfonyl]-1,3-thiazolidine-4-carboxylic acid;
(4R)-3-[(3-bromo-5-chloro-2-hydroxyphenyl)sulfonyl]-1,3-thiazolidine-4-carboxylic acid;
(4S)-3-[(3-chloro-5-fluoro-2-hydroxyphenyl)sulfonyl]-1,3-thiazolidine-4-carboxylic acid; and
(4R)-3-[(2-amino-5-chloro-4-methylphenyl)sulfonyl]-1,3-thiazolidine-4-carboxylic acid.

27. The compound of claim 12, wherein the compound of is (4R)-4-hydroxy-1-[(3,4,5-trichloro-2-hydroxyphenyl)sulfonyl]-L-proline.

28. The compound of claim 12, wherein the compound is 1-[(3,4,5-trichloro-2-hydroxyphenyl)sulfonyl]-L-proline.

29. The compound of claim 19, wherein the compound is (2S)-1-[(3,4,5-trichloro-2-hydroxyphenyl)sulfonyl]piperidine-2-carboxylic acid.

30. The compound of claim 23, wherein the compound is (4R)-3-[(3,4,5-trichloro-2-hydroxyphenyl)sulfonyl]-1,3-thiazolidine-4-carboxylic acid.

31. A pharmaceutical composition comprising a compound selected from one of claims 1, 12, 19, or 23, and a pharmaceutically acceptable carrier.

32. The pharmaceutical composition of claim 31, wherein the compound is (4R)-4-hydroxy-1-[(3,4,5-trichloro-2-hydroxyphenyl)sulfonyl]-L-proline.

33. The pharmaceutical composition of claim 31, wherein the compound is 1-[(3,4,5-trichloro-2-hydroxyphenyl)sulfonyl]-L-proline.

34. The pharmaceutical composition of claim 31, wherein the compound is (2S)-1-[(3,4,5-trichloro-2-hydroxyphenyl)sulfonyl]piperidine-2-carboxylic acid.

35. The pharmaceutical composition of claim 31, wherein the compound is (4R)-3-[(3,4,5-trichloro-2-hydroxyphenyl)sulfonyl]-1,3-thiazolidine-4-carboxylic acid.

36. A method of treating or preventing a hepatitis C infection in humans, which comprises administering an effective amount of a compound of formula (Ia): 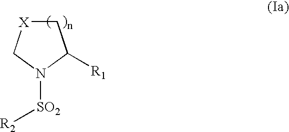
Wherein:
R₁ is H, —COOH, —CO₂R₄, cyano, tetrazole, a straight chain alkyl of 1 to 6 carbon atoms optionally substituted with OH, amine, or —COOH, an optionally substituted —C(O)-alkyl, or an optionally substituted —C(O)-aryl, wherein R₄ is an alkyl, aryl, cycloalkyl or a heteroaryl;
R₂ is an aryl or a heteroaryl group optionally substituted with one to five substituents selected from the group consisting of halogen, —NO₂, —CN, —N₃, —CHO, —CF₃, —OCF₃, —R₃, —OR₃, —S(O)ₘR₃, —NR₃R₃, —NR₃S(O)ₘR₃, —NR₃C(O)R₃, —C(O)R₃, —C(O)OR₃, —C(O)NR₃R₃, —OC(O)R₃, —OC(O)OR₃, —OC(O)NR₃R₃, NR₃C(O)R₃, —NR₃C(O)OR₃, and —NR₃C(O)NR₃R₃, wherein m is 0, 1, or 2;
R₃ is H, an alkyl of 1-6 carbon atoms, a branched alkyl of 1-8 carbon atoms, a cycloalkyl of 3 to 6 carbon atoms, phenyl, heteroaryl, an alkenyl of 2-6 carbon atoms, or an alkynyl of 2-6 carbon atoms;
X is CH₂, CHOR₃, or S; and
n is 1 or 2;
and all crystalline forms and pharmaceutically acceptable salts thereof.

37. The method of claim 36, wherein the compound is (4R)-4-hydroxy-1-[(3,4,5-trichloro-2-hydroxyphenyl)sulfonyl]-L-proline.

38. The method of claim 36, wherein the compound is 1-[(3,4,5-trichloro-2-hydroxyphenyl)sulfonyl]-L-proline.

39. The method of claim 36, wherein the compound is (2S)-1-[(3,4,5-trichloro-2-hydroxyphenyl)sulfonyl]piperidine-2-carboxylic acid.

40. The method of claim 36, wherein the compound is (4R)-3-[(3,4,5-trichloro-2-hydroxyphenyl)sulfonyl]-1,3-thiazolidine-4-carboxylic acid.
